(19) United States
(12) Patent Application Publication
(10) Pub. No.: US 2019/0364883 A1
(43) Pub. Date: Dec. 5, 2019

(54) AUTOMATIC BEARING DEVICE FOR LIVING CELL VITRIFICATION AND THAWING AND OPERATING SYSTEM THEREOF

(71) Applicant: Lijia YU, Zhaodong (CN)

(72) Inventor: Lijia YU, Zhaodong (CN)

(21) Appl. No.: 16/131,521

(22) Filed: Sep. 14, 2018

(30) Foreign Application Priority Data
Jun. 1, 2018 (CN) 201810550150.8

(51) Int. Cl. A01N 1/02; C12M 1/00

(52) U.S. Cl. CPC A01N 1/0252; C12M 45/22; C12M 45/20

(57) ABSTRACT

The automatic bearing device for living cell freezing and thawing includes a droplet changing area on one end and a sample bearing area communicating with the droplet changing area and arranged on the other end. The droplet changing area has a structure of a shallow dish for droplet change, and the bottom surface gradually inclines downwards from the free end to one end communicating with the sample bearing area. The sample bearing area has an elongated concave dish for containing frozen/thawed carriers. At least one part of the side of the droplet changing area is connected with the corresponding side of the sample bearing area. A sealer is arranged in the connection position, and a removable baffle is arranged above the connection position. The device realizes rapid and effective vitrification for living cell freezing and thawing.

### CROSS-REFERENCE TO RELATED APPLICATIONS

See Application Data Sheet.

### STATEMENT REGARDING FEDERALLY SPONSORED RESEARCH OR DEVELOPMENT

Not applicable.

### THE NAMES OF PARTIES TO A JOINT RESEARCH AGREEMENT

Not applicable.

### INCORPORATION-BY-REFERENCE OF MATERIAL SUBMITTED ON A COMPACT DISC OR AS A TEXT FILE VIA THE OFFICE ELECTRONIC FILING SYSTEM (EFS-WEB)

Not applicable.

### STATEMENT REGARDING PRIOR DISCLOSURES BY THE INVENTOR OR A JOINT INVENTOR

Not applicable.

### BACKGROUND OF THE INVENTION

### 1. Field of the Invention

The present invention belongs to the technical field of human tissue freezing and thawing, and particularly related to an automatic bearing device for living cell vitrification and thawing and operating system thereof.

### 2. Description of Related Art Including Information Disclosed Under 37 CFR 1.97 and 37 CFR 1.98.

The basic principle of the vitrification method is that cryoprotectant with high concentration is solidified in ultra-low temperature environment to form irregular vitrified solid, and the normal distribution of molecules and ions in the liquid state is kept, thereby playing a protective role when vitrification occurs in cells. The existing freezing technology can be used for cryopreservation of sperm, ova and embryos of different stages, and especially, the vitrification technology has been widely accepted in medical field and gradually supersedes the traditional slow-rate freezing method as the optimal choice of freezing technology.

The prior art mostly focuses on manual operation, specifically comprising: 1. dehydrating cells, and performing equalization treatment with cryoprotectant; 2. manually stretching and burning a glass tube to prepare a cytopipette, and making a thin tube; 3. manually sucking the liquid containing living cells with the thin tube from the cryoprotectant, applying the liquid to a flaky freezing carrier, and manually inserting the carrier in liquid nitrogen quickly after identifying the target cells and sucking the excess liquid to complete freezing. The manual operation method requires the time to be as short as possible and the coating to be as thin as possible to realize fast cooling. The operation often requires the operator to have proficient operation skill and to set the droplets to be the liquid with gradient concentration so as to gradually dehydrate the cells in the gradient liquid. The process requires fast operation, improper operations easily result in non-standard freezing of living cells due to overlarge osmotic pressure or failure to standardize the operation process, and the cells die or are lost very easily.

### BRIEF SUMMARY OF THE INVENTION

To solve the above technical problems, the present invention provides an automatic bearing device for living cell freezing and thawing to realize rapid and effective vitrification for living cell freezing and thawing.

The present invention has the following specific technical solution:

An automatic bearing device for living cell freezing and thawing, comprising a droplet changing area on one end and a sample bearing area communicating with the droplet changing area and arranged on the other end;
the droplet changing area has a structure of a shallow dish for droplet change, the bottom surface of the droplet changing area gradually inclines downwards from the free end to one end communicating with the sample bearing area, and a drainage part is arranged on the free end;
the sample bearing area is configured to have a structure of an elongated concave dish for containing frozen/thawed carriers; at least one part of the side of the droplet changing area communicating with the sample bearing area is connected with the corresponding side of the sample bearing area, the height of the top surface of the sample bearing area is flush with the bottom surface of the droplet changing area at the connection side, a sealer is arranged in the connection position, and a removable baffle is arranged above the connection position; and the effective interception height of the baffle is not lower than the highest value of the concave dish in the droplet changing area.

In an improved technical solution, the opposite side of the communicating side of the sample bearing area extends outwards to form a receiving part; the device further comprises a sealing sleeve having an elongated inner cavity, and an opening is formed on one side of the sealing sleeve and provided with a push-pull side door on the top and a locking mechanism for locking the side door and the sealing sleeve on the bottom; a sliding groove is arranged on the bottom of the inner cavity, and a sliding block matching with the sliding groove is arranged on the bottom of the sample bearing area; and the elongated sample bearing area can be pulled along the opening end in the inner cavity of the sealing sleeve.

Further, a side door locating frame is arranged on the side with the opening of the sealing sleeve and comprises an annular rectangular frame on the top, a bottom plate and two side plates, limiting columns retractable horizontally are arranged on both sides of the bottom of the annular rectangular frame, the side door is embedded in the annular rectangular frame, and the limiting columns extend to exactly butt against the bottom of the side door under the condition of not closing;
bulges are arranged on both sides located above the communicating side of the sample bearing area, and the bulges on both sides are respectively located on the same axis as the limiting columns;
first blocks are arranged on the inner walls of both sides of the side plates; both sides of the receiving part are provided with second blocks in an outward extension mode, and when the sampling bearing area is pulled to the outermost end, the first blocks exactly butt against the front sides of the second blocks.

Further, sealing gaskets are arranged at least on the inner side of the side door, and a locating rubber ball is respectively arranged on the upper edge and the lower edge of the inner side of the side door; the annular rectangular frame and the bottom plate are respectively provided with a locating slot; and when the side door is lowered, the locating rubber balls are exactly corresponding to the locating slots.

Further, the inclination angle of the bottom surface of the droplet changing area is 5-15°; and a clamping part is arranged on the top of the baffle in an extension mode.

Further, a convex part is arranged on the outer side of the sealing mechanism, and a notch is formed on the bottom of the droplet changing area and matches with the convex part;
an extension film is embedded in the inner bottom surface of the droplet changing area near the baffle, and the extension film bends and extends upwards over the baffle from the bottom surface and is a waterproof film.

Further, one side or both sides of the top of the baffle are provided with a connecting rod in an outward extension mode, and the lower part of the connecting rod is connected with a fixing rod; and insertion holes for inserting the fixing rod are formed on the connection side of the droplet changing area and both sides of the baffle.

The present invention further discloses an automatic operating system for living cell freezing and thawing, comprising at least:
an operating desk device, comprising an automatic bearing device storing mechanism, a transport mechanism and an operating desk mechanism, wherein the automatic bearing device storing mechanism is configured to have a storing cavity for storing the automatic bearing device, a tray is arranged below each automatic bearing device, an opening is formed on the lower end of the automatic bearing device storing mechanism, the lower part of the opening is connected with the end of the transport mechanism, and the opposite side of the opening of the automatic bearing device storing mechanism is also provided with a pushing mechanism for pushing the automatic bearing device below and the tray to the transport mechanism; the transport mechanism is formed by encircling conveying wheels with belt bodies on both sides, and both sides of the tray are exactly placed on the belt bodies on both sides of the conveying belt; and the operating desk mechanism is configured to place the tray and the automatic bearing device above;
a manipulator device, comprising a suspension arm and a manipulator arranged below the suspension arm, wherein the suspension arm is configured to have a structure which can be moved horizontally in X-axis direction, and the manipulator is configured to have a structure which can be moved in Y-axis direction and Z-axis direction;
a droplet changing device, comprising a bracket, a movable tube adaptor arranged on the bracket, a droplet inlet tube connected with the tube adaptor and arranged above the droplet changing area, a first droplet suction tube, and a second droplet suction tube arranged above the sample bearing area, wherein the tube adaptor is provided with an independent electric valve corresponding to each tube body, and the droplet inlet tube, the first droplet suction tube and the second droplet suction tube respectively control droplet suction through a liquid extraction pump;
a target cell sap transfer device, comprising a moving rack, a pipette arranged on the moving rack and used for sucking target cell sap, and a pump body providing suction force for the pipette;
a main control device, configured to control various operations of the operating desk device, the manipulator device, the droplet changing device and the target cell sap transfer device.

In an improved solution, a locating column is arranged in the middle of the bottom of the tray and has a structure of inverted isosceles trapezoid;
the operating desk mechanism is provided with a locating chuck for holding the locating column, the locating chuck is composed of a high stopper on the front side and a low stopper on the back side, and a space for holding the locating column is arranged between the high stopper and the low stopper; a vertical moving device is also arranged on the bottom of the locating chuck for adjusting the height of the locating chuck;
one side of the storing cavity of the automatic bearing device storing mechanism is provided with hackle marks, the other side thereof is vertically provided with adjusting tanks at intervals along the inner wall of the storing cavity, layering barrier strips extending outwards are embedded in the adjusting tanks, the top of each layering barrier strip located in the adjusting tank is provided with an elastic component connected with the inner top wall of the adjusting tank, and each layering barrier strip exactly butts against the tray of the corresponding layer;
when the tray and the automatic bearing device thereof are lowered, the layering barrier strip presses downwards by the gravity of the automatic bearing device and rotates with the contact point of the layering barrier strip and the adjusting tank notch as the axle center; the tray and the automatic bearing device thereof are moved down by one layer, and then the layering barrier strip butts against the bottom of the tray of the next layer under the recovery of the elastic component.

In an improved technical solution, the pipette is composed of a transparent tube body and a pipette needle arranged on the front end of the transparent tube body, the transparent tube body is provided with a measuring scale, an overflow port is arranged on the tube body at the highest scale level, and an overflow tank is arranged on the outer wall of the tube body corresponding to the lower part of the overflow port.

The present invention has the following beneficial effects: the present invention provides an automatic bearing device for living cell freezing and thawing and an automatic bearing device using the device. The automatic bearing device comprises a droplet changing part and a carrier freezing/thawing part, wherein the bottom surface of the droplet changing part is a inclined plane, the bottom surface of the carrier freezing/thawing part is the next step surface, the target frozen cells after droplet change directly flow into the carrier freezing/thawing part, the liquid is sucked, and freezing/thawing of living cells is completed. Compared with the prior art, the automatic bearing device is more accurate in operation, which avoids the loss of cells during the process of droplet change. Meanwhile, excess liquid can be removed by suction to avoid affecting the quick freezing and thawing of cells. The droplet changing part and the sample bearing part for freezing/thawing carriers are separated to prevent droplet changing operation from destroying carrier composition, and meanwhile, the carrier part is laid horizontally to make the target cells laid horizontally as much as possible so as to maximize the exposed surface area of the cells. In addition, the sample bearing area of the automatic bearing device of the present invention is integrated, and pushed into the sealing sleeve to be sealed for liquid nitrogen freezing, so the operation is convenient and contamination can be avoided.

The automatic operating system of the present invention realizes automatic operation through the operating desk device composed of the automatic bearing device storing mechanism, the transport mechanism and the operating desk mechanism, the operation is stable, the locating is accurate, and the safety and the efficiency are both greatly enhanced.

### BRIEF DESCRIPTION OF THE SEVERAL VIEWS OF THE DRAWINGS

To more clearly describe the technical solution in the embodiments of the present invention, the drawings to be used in the embodiments will be simply presented below.

FIG. 1 is a structural schematic view of one embodiment of an automatic bearing device for living cell freezing and thawing of the present invention.

FIG. 2 is a structural schematic view of the connection position of a droplet changing area and a sample bearing area.

FIG. 3 is a cross-sectional schematic view of a sample bearing area having a sealing sleeve.

FIG. 4 is a cross-sectional schematic view of a sealing sleeve.

FIG. 5 is a partially enlarged schematic view of Position A in FIG. 4.

FIG. 6 is a structural schematic view of combined use of a sample bearing area and a sealing sleeve of the present invention.

FIG. 7 is a cross-sectional schematic view of combined use of a sample bearing area and a sealing sleeve of the present invention.

FIG. 8 is another structural schematic view of the connection position of a droplet changing area and a sample bearing area.

FIG. 9 is a cross-sectional schematic view of the connection position of a droplet changing area and a sample bearing area shown in FIG. 8.

FIG. 10 is a structural schematic view of one embodiment of an automatic operating system of the present invention.

FIG. 11 is a partial structural schematic view of a diagram of automatic output of an automatic bearing device.

FIG. 12 is a structural schematic view of a locating device of an operating desk mechanism.

FIG. 13 is a structural schematic view of a pipette for quantitative suction.

### DETAILED DESCRIPTION OF THE INVENTION

The embodiments of the present invention are described below in combination with drawings. The description is only the exemplary description of the nature, and does not limit the present invention and the application and purpose thereof. For example, each embodiment of the present invention is expected to be widely used for living cells, including but not limited to embryos, sperm, ova, etc.

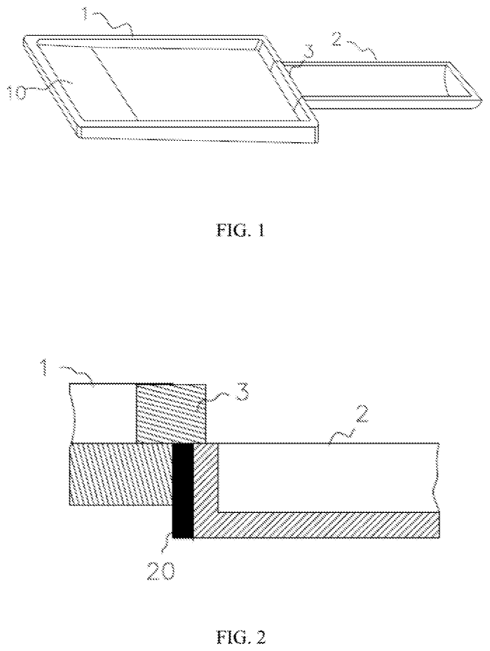

FIG. 1 shows one embodiment of the automatic bearing device for living cell freezing and thawing of the present invention.

The automatic bearing device comprises a droplet changing area 1 on one end and a sample bearing area 2 communicating with the droplet changing area 1 and arranged on the other end.

The droplet changing area 1 has a structure of a shallow dish for droplet change, the bottom surface of the droplet changing area 1 gradually inclines downwards from the free end to one end communicating with the sample bearing area 2, and a drainage part 10 is arranged on the free end.

The sample bearing area 2 is configured to have a structure of an elongated concave dish for containing frozen/thawed carriers; at least one part of the side of the droplet changing area 1 communicating with the sample bearing area 2 is connected with the corresponding side of the sample bearing area 2, the height of the top surface of the sample bearing area 2 is flush with the bottom surface of the droplet changing area 1 at the connection side, a sealing mechanism 20 is arranged in the connection position, as shown in FIG. 2, and a removable baffle 3 is arranged above the connection position; and the effective interception height of the baffle 3 is not lower than the highest value of the concave dish in the droplet changing area 1.

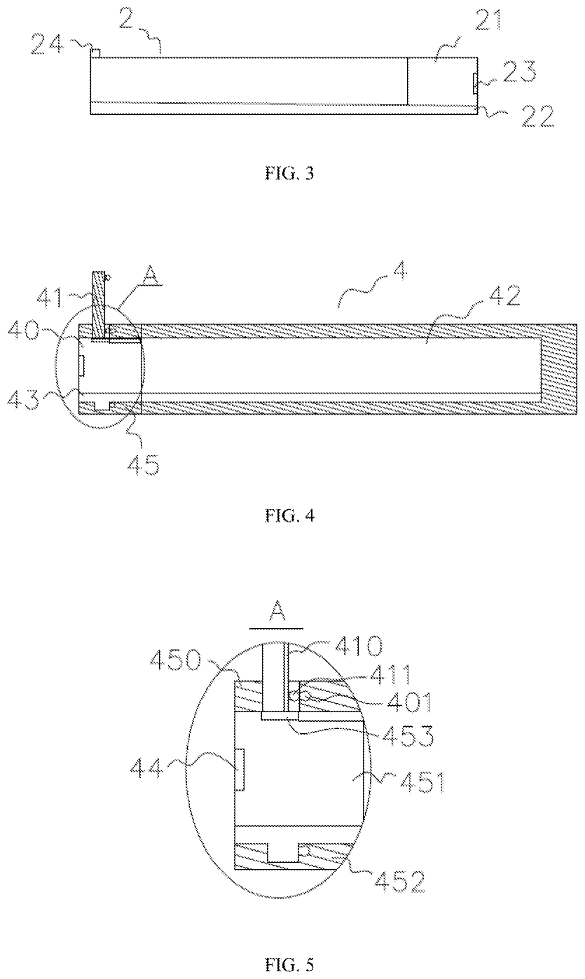

In some embodiments, the sample bearing area 2 is set to have a portable seal structure so that the operation of transferring into a liquid nitrogen container is safer and more convenient so as to avoid contamination. The specific solution of one embodiment is shown in FIG. 3-4. The opposite side end of the communicating side of the sample bearing area 2 extends outwards to form a receiving part 21; the device further comprises a sealing sleeve 4 having an elongated inner cavity 42, and an opening 40 is formed on one side of the sealing sleeve 4 and provided with a push-pull side door 41 on the top and a locking mechanism for locking the side door 41 and the sealing sleeve 4 on the bottom; a sliding groove 43 is arranged on the bottom of the inner cavity 42, and a sliding block 22 matching with the sliding groove 43 is arranged on the bottom of the sample bearing area 2; and the elongated sample bearing area 2 can be pulled along the opening 40 end in the inner cavity 42 of the sealing sleeve 4.

A side door locating frame 45 is arranged on the side with the opening 40 of the sealing sleeve 4 and comprises an annular rectangular frame 450 on the top, a bottom plate 451 and two side plates 452, limiting columns 453 retractable horizontally are arranged on both sides of the bottom of the annular rectangular frame 450, the side door 41 is embedded in the annular rectangular frame 450, and the limiting columns 453 extend to exactly butt against the bottom of the side door 41 under the condition of not closing.

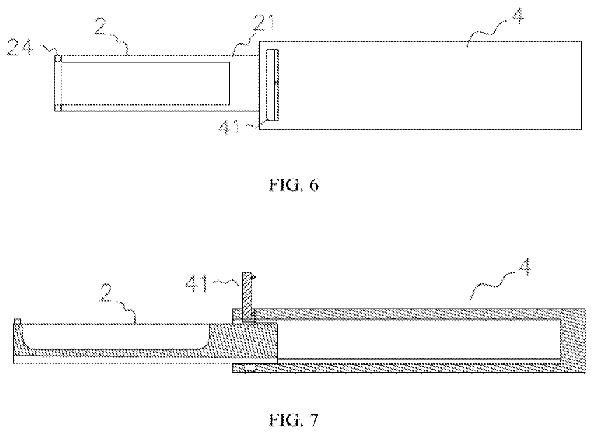

FIG. 6-7 are structural schematic diagrams of combined use of the sample bearing area and the sealing sleeve of the present invention. As shown in FIG. 7, bulges 24 are arranged on both sides located above the communicating side of the sample bearing area 2, and the bulges 24 on both sides are respectively located on the same axis as the limiting columns 453. First blocks are arranged on the inner walls of both sides of the side plates 452; both sides of the receiving part 21 are provided with second blocks 23 in an outward extension mode, and when the sampling bearing area 2 is pulled to the outermost end, the first blocks 44 exactly butt against the front sides of the second blocks 23.

Sealing gaskets 410 are arranged at least on the inner side of the side door 41, and a locating rubber ball 411 is respectively arranged on the upper edge and the lower edge of the inner side of the side door 41; the annular rectangular frame 450 and the bottom plate 451 are respectively provided with a locating slot 401; and when the side door 41 is lowered, the locating rubber balls 411 are exactly corresponding to the locating slots 401.

It should also be noted that the side door locating frame 45 is connected to the front side of the sealing sleeve in a removable mode, and the front side of the sample bearing area 2 is also provided with a magnet device. When the frozen cells enter the thawing mode, the side door locating frame 45 is removed, the sample bearing area 2 is adsorbed and pulled by the front end (iron) of the following manipulator device.

It should be noted that in some embodiments of the present invention, to reasonably set the inclination of the bottom surface of the droplet changing area 1, the inclination angle of the bottom surface of the droplet changing area 1 is set to 5-15°. To facilitate operation of the manipulator, a clamping part 30 is arranged on the top of the baffle 3 in an extension mode for clamping and taking out the baffle 3.

The baffle 3 of the present invention is set in a connection or embedded mode based on the criterion of realizing the barrier effect. In some concrete embodiments, as shown in FIG. 8, one side or both sides of the top of the baffle 3 are provided with a connecting rod 32 in an outward extension mode, and the lower part of the connecting rod 32 is connected with a fixing rod 33; and insertion holes 12 for inserting the fixing rod 33 are formed on the connection side of the droplet changing area 1 and both sides of the baffle 3.

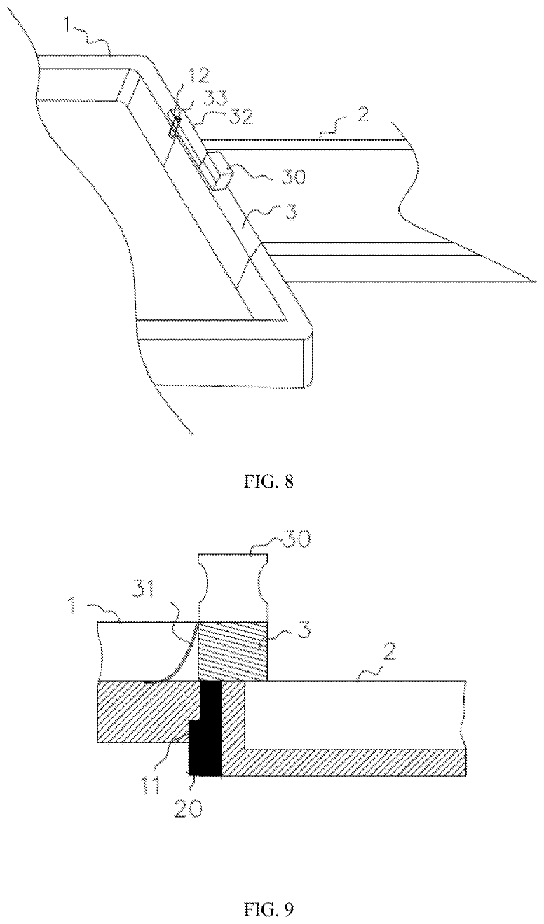

It should also be noted that to make the connection of the droplet changing area and the sample bearing area more closer, in some embodiments, the interface between the droplet changing area and the sample bearing area is set as matching of a notch and a lug; as shown in FIG. 9, a convex part is arranged on the outer side of the sealing mechanism 20, and a notch 11 is formed on the bottom of the droplet changing area 1 and matches with the convex part; an extension film 31 is embedded in the inner bottom surface of the droplet changing area 1 near the baffle 3, and the extension film 31 bends and extends upwards over the baffle 3 from the bottom surface and is a waterproof film. When the baffle 3 is pulled out, the extension film 31 extends towards the sample bearing area 2 along the connection position under the impact of liquid flow of the droplet changing area to cover the connection seam so as to prevent the liquid from infiltrating into the seam.

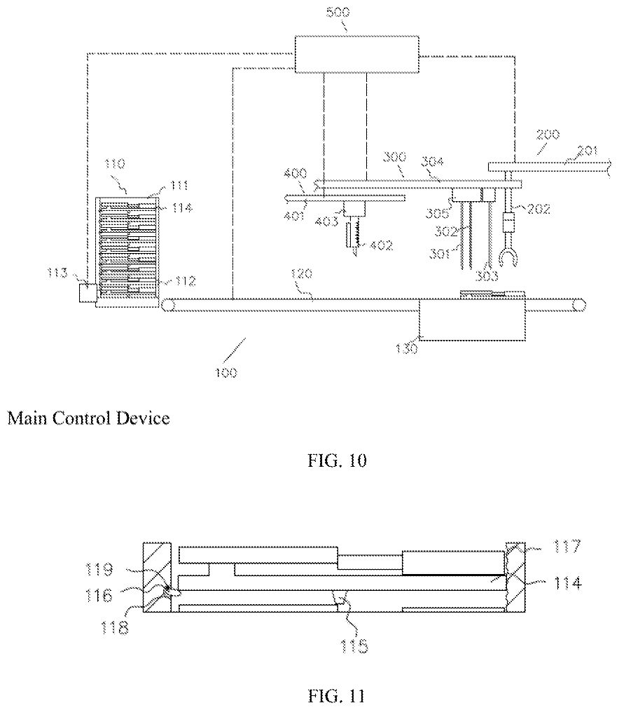

The present invention further discloses an automatic operating system based on the above automatic bearing device for living cell freezing and thawing. FIG. 10 shows one embodiment of the system, and specifically, the system comprises an operating desk device 100, a manipulator device 200, a droplet changing device 300, a target cell sap transfer device 400 and a main control device 500.

The operating desk device 100 comprises an automatic bearing device storing mechanism 110, a transport mechanism 120 and an operating desk mechanism 130, wherein the automatic bearing device storing mechanism 110 is configured to have a storing cavity 111 for storing the automatic bearing device, a tray 114 is arranged below each automatic bearing device, an opening 112 is formed on the lower end of the automatic bearing device storing mechanism 110, the lower part of the opening 112 is connected with the end of the transport mechanism 120, and the opposite side of the opening of the automatic bearing device storing mechanism 110 is also provided with a pushing mechanism 113 for pushing the automatic bearing device below and the tray 114 to the transport mechanism 120; the transport mechanism 120 is formed by encircling conveying wheels with belt bodies on both sides, and both sides of the tray 114 are exactly placed on the belt bodies on both sides of the conveying belt 121; and the operating desk mechanism 130 is configured to place the tray 114 and the automatic bearing device above.

The manipulator device 200 comprises a suspension arm 201 and a manipulator 202 arranged below the suspension arm 201, wherein the suspension arm 201 is configured to have a structure which can be moved horizontally in X-axis direction, and the manipulator 202 is configured to have a structure which can be moved in Y-axis direction and Z-axis direction. The structure of three-axis operation, which can adopt the prior art, will not be described in detail herein.

The droplet changing device 300 comprises a bracket 304, a movable tube adaptor 305 arranged on the bracket 304, a droplet inlet tube 301 connected with the tube adaptor 305 and arranged above the droplet changing area 1, a first droplet suction tube 302, and a second droplet suction tube 303 arranged above the sample bearing area 2, wherein the tube adaptor 305 is provided with an independent electric valve corresponding to each tube body, and the droplet inlet tube 301, the first droplet suction tube 302 and the second droplet suction tube 303 respectively control droplet suction through a liquid extraction pump.

The target cell sap transfer device 400 comprises a moving rack 401, a pipette 402 arranged on the moving rack 401 and used for sucking target cell sap, and a pump body 403 providing suction force for the pipette 402;

The main control device 500 is configured to control various operations of the operating desk device 100, the manipulator device 200, the droplet changing device 300 and the target cell sap transfer device 400. For example, various controls on the droplet changing device 300—opening/closing of the droplet input tube 301, the first droplet suction tube 302 and the second droplet suction tube 303 are sent through the main control device 500, and the opening degree is controlled to realize the flow of cell droplet culture solution so as to realize standardized control of cell liquid environment.

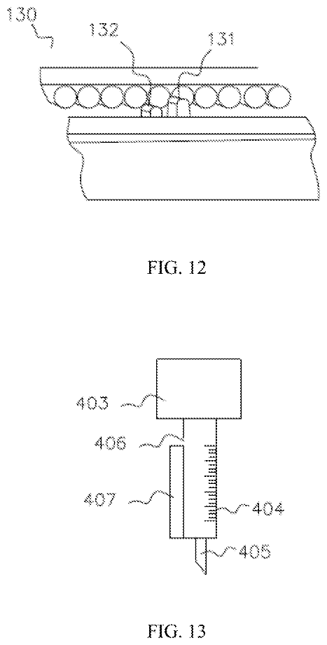

FIG. 11-12 show an improved solution for automatic input and locating of the automatic bearing device based on the above technical solutions.

As shown in FIG. 11, one side of the storing cavity 111 of the automatic bearing device storing mechanism 110 is provided with hackle marks 117, the other side thereof is vertically provided with adjusting tanks 118 at intervals along the inner wall of the storing cavity 111, layering barrier strips 116 extending outwards are embedded in the adjusting tanks 118, the top of each layering barrier strip 116 located in the adjusting tank 118 is provided with an elastic component 119 connected with the inner top wall of the adjusting tank 118, and each layering barrier strip 116 exactly butts against the tray 114 of the corresponding layer.

When the tray 114 and the automatic bearing device thereof are lowered, the layering barrier strip 116 presses downwards by the gravity of the automatic bearing device and rotates with the contact point of the layering barrier strip 116 and the adjusting tank 118 notch as the axle center; the tray 114 and the automatic bearing device thereof are moved down by one layer, and then the layering barrier strip 116 butts against the bottom of the tray 114 of the next layer under the recovery of the elastic component 119.

As shown in FIG. 11, a locating column 115 is arranged in the middle of the bottom of the tray 114 and has a structure of inverted isosceles trapezoid. As shown in FIG. 12, the operating desk mechanism 130 is provided with a locating chuck for holding the locating column 115, the locating chuck is composed of a high stopper 131 on the front side and a low stopper 132 on the back side, and a space for holding the locating column 115 is arranged between the high stopper 131 and the low stopper 132; a vertical moving device which is not shown is also arranged on the bottom of the locating chuck for adjusting the height of the locating chuck.

In some embodiments, the quantitative suction function of the pipette is improved, as shown in FIG. 13, the pipette 402 is composed of a transparent tube body 404 and a pipette needle 405 arranged on the front end of the transparent tube body 404, the transparent tube body 404 is provided with a measuring scale, an overflow port 406 is arranged on the tube body at the highest scale level, and an overflow tank 407 is arranged on the outer wall of the tube body corresponding to the lower part of the overflow port 406.

When the main control device 500 sends a control instruction of the cell freezing mode, the automatic bearing device storing mechanism 110 of the operating desk device 100 lowers the tray 114 with the automatic bearing device, the tray 114 is conveyed to the operating desk mechanism 130 by the transport mechanism 120, and because the operating desk of the operating desk mechanism 130 is provided with a locating point, the tray 114 is conveyed to the locating point. The pipette 402 sucks and transfers the target cell droplet to the droplet changing area 1 of the automatic bearing device according to the received instruction, and then the liquid changing operation for cell freezing is performed through the first droplet suction tube 302. It should be noted that the input end of the droplet input tube 301 is not limited to a pipette and also can be provided with a special cryoprotectant pipette so that the droplet input tube 301 also can be used for adding cryoprotectant. It should also be noted that the positions for target cell sap input, droplet input and droplet suction of the present invention can be realized respectively by locating the moving rack 401 and the bracket 304, i.e. the bracket position is fixed, the tray position is fixed, and the operation position of each droplet input tube or suction tube is basically fixed. Subsequently, the manipulator device 200 take outs the baffle 3, the cell droplets in the droplet changing area 1 are transferred to the sample bearing area 2 laid with freezing carriers, the flow process for transfer in the sample bearing area 2 makes the cryoprotectant droplets fuse faster until the cell droplets and the cryoprotectant achieve an equilibrium state, and the second droplet suction tube 303 can be used to suck the liquid adsorbed around the cells to maximize the exposed surface area of the cells. Finally, the cells are moved from the droplet changing area 1 by the manipulator device, and the sample bearing area 2 is pushed into the sealing sleeve 4; when the side door 41 is lowered, the locating rubber balls 411 is pushed into the locating slots 401 to realize sealing, and then the cells are quickly transferred into the freezing medium to complete freezing.

The steps of thawing operation are in the inverse sequence of that of freezing operation. The sample bearing area is pulled out of the sealing sleeve, placed in the corresponding position of the tray, and transferred to the operating desk position. Meanwhile, the droplet input tube 301 and the first droplet suction tube 302 of the droplet changing device are adjusted to a second fixed point position (i.e. located above the sample bearing area), then the droplet changing operation for cell thawing is performed according to the instruction until the cells reach the specified culture solution equilibrium state, thawing is completed, and the cell droplets are transferred to cultivating carriers through the pipette 402.

The above embodiments only express several implementation modes of the present invention, and are described more specifically in details, but shall not be consequently interpreted as a limitation to the scope of the patent for the present invention. It should be noted that, for those ordinary skilled in the art, several deformations and improvements can also be made without departing from the concept of the present invention, all of which belong to the protection scope of the present invention. Therefore, the protection scope of the patent for the present invention shall be subject to appended claims.

1. An automatic bearing device for living cell freezing and thawing, comprises:
a droplet changing area on one end; and
a sample bearing area communicating with the droplet changing area and arranged on another end;
wherein said droplet changing area has a structure of a shallow dish for droplet change,
wherein the bottom surface of the droplet changing area gradually inclines downwards from the free end to one end communicating with the sample bearing area, and
wherein a drainage part is arranged on the free end,
wherein the sample bearing area has a structure of an elongated concave dish for containing frozen/thawed carriers,
wherein at least one part of the side of the droplet changing area communicating with the sample bearing area is connected with the corresponding side of the sample bearing area,
wherein the height of the top surface of the sample bearing area is flush with the bottom surface of the droplet changing area at the connection side,
wherein a sealing mechanism is arranged in the connection position,
wherein a removable baffle is arranged above the connection position, and
wherein the effective interception height of the baffle is not lower than the highest value of the concave dish in the droplet changing area.

2. The automatic bearing device for living cell freezing and thawing of claim 1, wherein the opposite side end of the communicating side of the sample bearing area extends outwards to form a receiving part, further comprising:
a sealing sleeve having an elongated inner cavity,
an opening being formed on one side of the sealing sleeve and provided with a push-pull side door on the top, and
a locking mechanism for locking the side door and the sealing sleeve on the bottom,
a sliding groove arranged on the bottom of the inner cavity, and
a sliding block matching with the sliding groove being arranged on the bottom of the sample bearing area,
wherein the elongated sample bearing area is pulled along the opening end in the inner cavity of the sealing sleeve.

3. The automatic bearing device for living cell freezing and thawing of claim 2, further comprising:
a side door locating frame being arranged on the side with the opening of the sealing sleeve and being comprised of an annular rectangular frame on the top, a bottom plate and two side plates;
limiting columns retractable horizontally being arranged on both sides of the bottom of the annular rectangular frame, the side door being embedded in the annular rectangular frame, and the limiting columns extending to exactly butt against the bottom of the side door under the condition of not closing;
bulges being arranged on both sides located above the communicating side of the sample bearing area, the bulges on both sides being respectively located on the same axis as the limiting columns; and
first blocks being arranged on the inner walls of both sides of the side plates, wherein both sides of the receiving part are provided with second blocks in an outward extension mode, and when the sampling bearing area is pulled to the outermost end, the first blocks exactly butt against the front sides of the second blocks.

4. The automatic bearing device for living cell freezing and thawing of claim 3, further comprising:
sealing gaskets arranged at least around the inner side of the side door; and
a locating rubber ball respectively arranged on the upper edge and the lower edge of the inner side of the side door,
wherein the annular rectangular frame and the bottom plate are respectively provided with a locating slot, and
wherein, when the side door is lowered, the locating rubber balls are exactly corresponding to the locating slots.

5. The automatic bearing device for living cell freezing and thawing of claim 1, wherein inclination angle of the bottom surface of the droplet changing area is 5-15°, further comprising:
a clamping part arranged on the top of the baffle in an extension mode.

6. The automatic bearing device for living cell freezing and thawing of claim 1, further comprising:
a convex part arranged on the outer side of the sealing mechanism; and
a notch formed on the bottom of the droplet changing area and matches with the convex part; and
an extension film embedded in the inner bottom surface of the droplet changing area near the baffle, the extension film bending and extending upwards over the baffle from the bottom surface and is a waterproof film.

7. The automatic bearing device for living cell freezing and thawing of claim 1, wherein at least one side of the top of the baffle is provided with a connecting rod in an outward extension mode, and wherein the lower part of the connecting rod is connected with a fixing rod, and wherein insertion holes for inserting the fixing rod are formed on the connection side of the droplet changing area and both sides of the baffle.

8. An automatic operating system for living cell freezing and thawing, comprising:
an operating desk device being comprised of an automatic bearing device storing mechanism, a transport mechanism and an operating desk mechanism,
wherein the automatic bearing device storing mechanism comprises a storing cavity for storing the automatic bearing device, a tray arranged below each automatic bearing device, and an opening formed on the lower end of the automatic bearing device storing mechanism,
wherein the lower part of the opening is connected with the end of the transport mechanism, and
wherein the opposite side of the opening of the automatic bearing device storing mechanism is provided with a pushing mechanism for pushing the automatic bearing device below and the tray to the transport mechanism
wherein the transport mechanism is formed by encircling conveying wheels with belt bodies on both sides, wherein both sides of the tray are exactly placed on the belt bodies on both sides of the conveying belt, and wherein the operating desk mechanism is configured to place the tray and the automatic bearing device above;
a manipulator device being comprised of a suspension arm and a manipulator arranged below the suspension arm, wherein the suspension arm has a structure which can be moved horizontally in X-axis direction, and wherein the manipulator has a structure which can be moved in Y-axis direction and Z-axis direction;
a droplet changing device being comprised of a bracket, a movable tube adaptor arranged on the bracket, a droplet inlet tube connected with the tube adaptor and arranged above the droplet changing area, a first droplet suction tube, and a second droplet suction tube arranged above the sample bearing area,
wherein the tube adaptor is provided with an independent electric valve corresponding to each tube body, and
wherein the droplet inlet tube, the first droplet suction tube and the second droplet suction tube respectively carry out the operation of controlling droplet change through a liquid extraction pump;
a target cell sap transfer device being comprised of a moving rack, a pipette arranged on the moving rack and used for sucking target cell sap, and a pump body providing suction force for the pipette; and
a main control device, configured to control various operations of the operating desk device, the manipulator device, the droplet changing device and the target cell sap transfer device.

9. The automatic operating system for living cell freezing and thawing of claim 8, further comprising:
a locating column being arranged in the middle of the bottom of the tray and having a structure of inverted isosceles trapezoid;
a locating chuck for holding the locating column, the locating chuck being comprised of a high stopper on the front side and a low stopper on the back side, and a space for holding the locating column arranged between the high stopper and the low stopper; and
a vertical moving device arranged on the bottom of the locating chuck for adjusting the height of the locating chuck,
wherein one side of the storing cavity of the automatic bearing device storing mechanism is provided with hackle marks,
wherein the other side thereof is vertically provided with adjusting tanks at intervals along the inner wall of the storing cavity,
wherein layering barrier strips extending outwards are embedded in the adjusting tanks,
wherein the top of each layering barrier strip located in the adjusting tank is provided with an elastic component connected with the inner top wall of the adjusting tank,
wherein each layering barrier strip exactly butts against the tray of the corresponding layer,
wherein, when the tray and the automatic bearing device thereof are lowered, the layering barrier strip presses downwards by the gravity of the automatic bearing device and rotates with the contact point of the layering barrier strip and the adjusting tank notch as the axle center,
wherein the tray and the automatic bearing device thereof are moved down by one layer, and
wherein, then, the layering barrier strip butts against the bottom of the tray of the next layer under the recovery of the elastic component.

10. The automatic operating system for living cell freezing and thawing of claim 8, further comprising:
a pipette being comprised of a transparent tube body and a pipette needle arranged on the front end of the transparent tube body, the transparent tube body being provided with a measuring scale, an overflow port being arranged on the tube body at the highest scale level, and an overflow tank being arranged on the outer wall of the tube body corresponding to the lower part of the overflow port.
